(19) United States
(12) Patent Application Publication
(10) Pub. No.: US 2022/0175299 A1
(43) Pub. Date: Jun. 9, 2022

(54) System for Predicting at Least One Cardiological Dysfunction in an Individual

(71) Applicant: Albert-Ludwigs-Universitat Freiburg, Freiburg (DE)

(72) Inventors: Amir JADIDI, Freiburg (DE); Thomas ARENTZ, Freiburg (DE); Björn MÜLLER-EDENBORN, Freiburg (DE)

(21) Appl. No.: 17/436,852

(22) PCT Filed: Mar. 5, 2020
(86) PCT No.: PCT/EP2020/055845

§ 371 Date: Sep. 7, 2021

(30) Foreign Application Priority Data
Mar. 8, 2019 (DE) 10 2019 203 155.2

(51) Int. Cl. A61B 5/353; A61B 5/355; A61B 5/366; A61B 5/333; A61B 5/30; A61B 5/308

(52) U.S. Cl. CPC A61B 5/353; A61B 5/355; A61B 5/366; A61B 2562/222; A61B 5/30; A61B 5/308; A61B 5/333

(57) ABSTRACT

A system is described for predicting at least one cardiological dysfunction in an individual, having a means for providing an ECG which has a number n of time-synchronized ECG traces, each comprising a chronological sequence of time signals representing a sinus rhythm of the individual's heartbeat, to which at least one P wave, a QRS complex and a T wave can be assigned in chronological order. A selection means selects at least two ECG traces from the n ECG traces, an analysis unit analyses the selected ECG traces as follows: a) determining an isoelectric signal level, b) determining a first point in time chronologically before the QRS complex, c) determining a second point in time chronologically after the first point in time and chronologically before the QRS complex, d) carrying out the determining steps a) to c) for all selected ECG traces, e) determining an earliest first point in time from all the first points in time determined for the respective selected ECG traces and a latest second point in time from all the second points in time determined for the respective selected ECG traces, f) determining a time interval delimited by the earliest first point in time and latest second point in time.

### CROSS-REFERENCE TO RELATED APPLICATIONS

Reference is made to PCT/EP2020/055845 filed Mar. 3, 2020, and German Patent Application No. 10 2019 203 155.2 filed Mar. 8, 2019, which are incorporated herein by reference in their entirety.

### BACKGROUND OF THE INVENTION

### Field of the Invention

The invention relates to a system for predicting at least one cardiological dysfunction in an individual.

### Description of the Prior Art

Medical specialists are of the general opinion that patients with an advanced atrial cardiomyopathy are at increased risk of developing cardiovascular disease which may typically occur in the form of auricular fibrillation, cardiac insufficiency or ischaemic stroke. At least one of mechanical and electrical malfunctions of the heart muscle occur in these cases in the region of the atrium stem from a pathological proliferation of the connective tissue (fibrosis) and an associated localized scarring of the heart muscle. In addition, a dilation of the atrium may also be presented. Current diagnostic techniques for detecting a patient suffering from atrial cardiomyopathy are based on either magnetic resonance-based atrial sequence imaging or on invasive electroanatomical mapping using a special 3D system from a cardiac electrophysiological catheter laboratory. Both diagnostic options are characterized by a limited availability and high costs. In the case of magnetic resonance-based investigations, moreover, gadolinium has to be injected as a contrast agent. For these reasons, it is obvious that investigations of this type in patients with atrial cardiomyopathy can only be carried out in exceptional cases, i.e. not systematically.

Because the pathology of atrial cardiomyopathy, in particular in the form of heart fibroses, is closely linked to an increased risk of cardiac arrhythmia, for example in the form of atrial fibrillation or atrial flutter, alternative cost-effective and resource-friendly diagnostic methods which could be used as low-cost predictive screening methods are desirable.

On the basis of detailed investigations into electrocardiograms which have been recorded for a plurality of healthy people as well as people with atrial cardiomyopathy, some ECG-based measurement parameters could be detected on the basis of which a qualified assessment of the risk of at least one of atrial fibrillation and stroke appears to be possible.

In an article by Jane Caldwell et al, “Prolonged P-wave Duration is Associated with Atrial Fibrillation Recurrence after Successful Pulmonary Vein Isolation for Paroxysmal Atrial Fibrillation”, Journal of Interventional Cardiac Electrophysiology, March 2014, Volume 39, Issue 2, pp. 131-138, it is stated that in the context of post-operative examinations of patients with pulmonary vein isolation, it has been confirmed that the P wave of an electrocardiogram (ECG) representing the sinus rhythm of the heart function, in particular its P wave duration, can be taken to be a relevant evaluation parameter for a risk assessment regarding the occurrence of atrial fibrillation or stroke. Thus, in the context of statistical evaluation, it could be concluded that patients with a prolonged P wave duration of ≥140 ms run a significantly higher risk of atrial fibrillation and/or stroke.

A more recent study by Yao-Sheng Wang et al., “Prolonged P-wave Duration is Associated with Atrial Fibrillation Recurrence after Radiofrequency Catheter Ablation: A Systematic Review and Meta-Analysis”, International Journal of Cardiology, Volume 227, 15 Jan. 2017, pp. 355-359, confirmed this in a meta-analysis of 9 separate studies. Thus, the electrocardiograms from a total of more than 1000 patients who had undergone a cardiological high frequency catheter ablation were analysed as regards a prolonged P wave duration. In the context of these investigations, it was shown that the probability of patients with a P wave duration of more than 149.5 ms of suffering from atrial fibrillation was 66%.

A study presented in the article by Amir Jadidi et al., “The Duration of the Amplified Sinus-P-Wave Identifies Presence of Left Atrial Low Voltage Substrate and Predicts Outcome After Pulmonary Vein Isolation in Patients With Persistent Atrial Fibrillation”, JACC: Clinical Electrophysiology, Volume 4, Issue 4, April 2018, 531-543, which was carried out on patients with pulmonary vein isolation, confirmed the relationship between a chronologically prolonged P wave and the increased risk of the recurrence of cardiac arrhythmia in the form of atrial fibrillation and stroke. The present study shows for the first time that the greatly amplified P wave duration, which can be deduced from a digitally amplified 12-lead surface ECG within a sinus rhythm, correlates very well with the extent of fibrotic low voltage substrate within the left atrium. Thus, patients with a P wave duration of ≥150 ms have an increased risk of the recurrence of arrhythmia.

### SUMMARY OF THE INVENTION

The invention is a system for predicting at least one cardiological dysfunction, in particular in the form of at least one of atrial fibrillation, ischaemic stroke and cardiac insufficiency, so that the reliability and robustness of a prediction regarding the probability that an individual will suffer a cardiac dysfunction can be significantly improved. In this manner, it will be possible to carry out systemic investigations on a large number of individuals in a rapid and economical manner in order, as a result, to make positive risk assessments regarding the occurrence of future cardiological dysfunction with a reliability for each individual of at least 80%, preferably at least 90%.

The invention is based on the realisation that the reliability with which a prediction of a potential risk to an individual of future atrial fibrillation or an ischaemic stroke is made is directly correlated to the accuracy of the metrologically detected P wave duration, the “Area under the total or left atrial P-wave (AUP), the quotient of the amplitude of the total P wave to the total P wave duration as well as the quotient of the mean or maximum amplitude of a pre-specified fraction of the P wave to the duration of this fraction or to the total duration of the P wave”.

With the aid of meticulous assessments of electrocardiograms which have been obtained from a large number of people, on the one hand the difficulties and the associated possibilities of errors in the risk assessment of cardiac arrhythmia have been detected on the basis of the P wave duration, and on the other hand it has been recognised that the strength of a risk assessment of this type is highly sensitive to the accuracy of the metrological detection of the P wave duration. Thus, the P wave duration has to be accurately determined with a maximum chronological error of ±2 ms, preferably a maximum of ±1 ms, particularly preferably a maximum of ±0.5 ms. If these tolerances are greater, the reliability of the prediction deteriorates in a non-linear manner. The quality of measurement forming the basis of the system of the invention for chronological detection of the P wave duration enables predictions which are specific to the individual to be made regarding future cardiovascular dysfunction with a probability of at least 80% right up to more than 90%.

In this regard, the system of the invention comprises a means for the preparation of an ECG recorded for an individual, which has a number n of time-synchronized ECG traces which each comprise a chronological sequence of time signals representing the sinus rhythm of a heartbeat of the individual. Preferably, the means for the preparation of the ECG is in the form of a digital 12-lead standard ECG recording device or in the form of a body surface ECG recording system which uses multiple electrodes in order to record the electrical cardiac stimuli. Alternatively, the means for the preparation of the n time-synchronized ECG traces may be configured only in the form of a storage medium on which the n ECG traces are stored in digital form. In a simplest form, it is also possible for only n=3 leads to be required which are the basis of a further evaluation.

Preferably, the n=12 time-synchronized ECG traces comprise, in a periodic sequence, the electrocardiographically recorded electrical activities of all of the heart muscle fibers which are respectively described as the sinus rhythm, the chronological sequence of which can be divided into what is known as the P wave, the QRS complex as well as the T wave. What is vitally important in the present case is the time interval for the P wave which corresponds to atrial stimulation and the start of which is initiated by an electrical stimulation in the sinus node. The electrical stimulation spreads from the sinus node in the right atrium via the left atrium in the direction of the AV node (atrioventricular node). The chronological end of the P wave is defined as reaching complete electrical stimulation of the atrium, which as a rule is chronologically before the ventricular stimulation defined by the QRS complex in the sinus rhythm.

In addition to the means for the preparation of an ECG recorded for an individual, the system comprises a selection means which selects that ECG trace from the number n of prepared ECG traces which are preferential for a P wave duration determination.

In the context of the processor-implemented selection means, the ECG traces in the digital form enable the use of a program which analyses at least one of image data and of digital analysis algorithms which, with the additional use of stored specialist information, for example in the form of reference data, as well as the optional use of self-learning data signal analysis routines, select those ECG traces from the n ECG traces which contain characteristic and complete P wave fractions. Out of all of the ECG traces, at least two or, as along as the selection criteria have been satisfied, preferably all of the electrocardiogram traces are selected. In this regard, the selection of suitable ECG traces is based on a specific degree of similarity between the ECG traces from the preparation means and a predetermined reference as the first decision criterion.

A processor-based computing unit which acts as an analysis unit and which communicates with the selection means analyses the pre-selected ECG traces as regarding the chronological starting and the chronological end of the P wave in at least one sinus rhythm per ECG trace.

In this regard, first, an isoelectric signal level has to be determined using the time signals from a selected ECG trace. An isoelectric signal level which can be associated with the time signals of a selected ECG trace is preferably characterized when, within a predetermined chronological interval, the ECG time signals have no technically assessable signal levels which are above the noise. In order to determine the isoelectric significant level, a time interval within a sinus rhythm which is chronologically before the P wave of at least 10 ms, preferably at least 20 ms, particularly preferably at least 30 ms, is suitable.

For the purposes of determining the start of the P wave, the analysis unit determines that first point in time which is chronologically before the QRS complex and from which the following time signals of the ECG trace have a signal level which deviates from the isoelectric signal level. Preferably, this signal level which deviates from the isoelectric signal level is at least twice the signal level of the isoelectric signal level.

Optionally, a further criterion for the determination of the first point in time defining the start of the P wave may be applied, in accordance with which respectively subsequent time signals have a positive increasing signal level within a first time interval immediately adjacent to the first point in time, that is the P wave defined by the time signals within the second point in time mathematically has a positive first derivative.

In order to determine the second point in time defining the end of the P wave, which is chronologically after the first point in time and before the QRS complex, that point in time is determined at which starting from a signal level which deviates from the isoelectric signal level, the time signals of the ECG trace go back to the isoelectric signal level. Advantageously, the end of the P wave, that is the second point in time, is taken to be that point in time the associated time signal corresponding to the isoelectric signal level and the level of the time signal immediately preceding this time signal is at least twice the signal level compared with the isoelectric signal level.

Advantageously, a second decision criterion which determines the second point in time may be used, in accordance with which the time signals within a second time interval adjacent to the second point in time must be at the isoelectric signal level or are delimited by the chronological start of the chronologically following QRS complex which corresponds to what is known as the ventricular complex. The second time interval should be at least 4 ms.

A further optional decision criterion which may be used in combination with or as an alternative to the aforementioned decision criteria for establishing the respective first and second points in time, which delimit the P wave chronologically, uses the comparison of the ECG traces with a reference time signal model of a sinus rhythm P wave. In the context of the comparison, a digital pattern recognition is carried out.

Out of all of the selected ECG traces which comprise respective time-synchronised time signals, the analysis unit selects the first point in time which is chronologically detected to be the earliest. In contrast, out of the selected ECG time signals, the second point in time which is selected is that which has been determined to be the chronologically latest second point in time. On the basis of the respective earliest first point in time as well as the respective latest second point in time, the analysis unit determines the actual (maximum) P wave duration which for further considerations corresponds to an exact duration for measurement of the P wave within the cardiac sinus rhythm.

The system of the furthermore comprises a comparator which determines a discrepancy between the determined P wave duration and a reference value, and generates a signal based on a second decision criterion. The second decision criterion is based on a maximum time interval Δtmax for which: 100≤Δtmax≤140 ms. In the case in which the determined P wave duration is more than Δtmax, the comparator generates the signal in the form of an acoustic, visual or haptically perceptible signal.

As already mentioned, the use of a 12-lead ECG permits 12×traces to be made which correspond to the standard 12-lead lead according to Einthoven, Goldberger and Wilson and which have the following designations: I, II, III, aVR, aVL, aVF, V1 to V6.

It has been shown to be particularly advantageous for the determination of the respective first point in time to select at least two, preferably all of the following ECG traces: II, III, aVF, aVR, V1, V2.

In order to determine the respective second point in time, advantageously, at least two, preferably all of the following ECG traces are suitable: I, II, III, aVR, aVL, aVF, V2 to V6.

According to the invention, it is also possible to use at least three ECG traces only in order to determine the P wave duration with a sufficiently high reliability. In this case, the ECG electrodes are disposed in a manner such that the measurement of at least one inferior or a lateral or an inferolateral or a superolateral or an anterior ECG trace is possible. In order to lead the lateral or inferior or inferolateral or a superolateral ECGs, the ECG electrodes may be applied to the right and left of the sternum or from the mid-sagittal plane of the body. In order to trace the anterior or posterior ECG, the ECG electrodes may be applied to the front and back of the thorax. In all cases, the ECG electrodes must respectively lie on opposite sides of the zero potential line of the field of the heart according to Augustus Waller (1887).

The separation of the two electrodes should be more than 1 cm in order to record the cardiac signals sufficiently and to make a diagnosis of an atrial cardiomyopathy accessible, for example: application to the right and left of the sternum or at the right and left clavicle (collar bone) or on the right ear and the left hand side clavicle (respectively on the other side of the zero potential line), or on the right hand and left hand.

In order to reduce artefacts as well as other types of perturbing influences, for example concerning the noise levels, during the evaluation of the ECG traces, it is proposed that the time signals of an ECG trace of at least two, preferably 10 to 1000 sinus rhythms of the heartbeat be overlaid and mathematically averaged. Overlaying and mathematical averaging is carried out by the analysis unit on all of the selected ECG traces separately. On the basis of the overlaid and averaged time signals per ECG trace, the analysis unit carries out the aforementioned determination steps in order to obtain the first and second points in time.

Optionally again, an integrator is provided which generates an integrated value on the basis of the determined P wave duration, what is known as the area under the curve value, over the chronological sequence of the time signals within the determined P wave duration. The comparator which is already available or a further additional comparator compares the determined integrated value with a reference value and generates a signal on the basis of a third decision criterion.

The quotient of the mean or maximum amplitude of the total P wave to the total P wave duration as well as the quotient of the mean or maximum amplitude of a pre-specified fraction of the P wave to the duration of that fraction or to the total duration of the P wave may be determined from the P wave determined as above. These parameters are compared with a reference value using the available comparator or a further comparator, which produces a signal on the basis of a further decision criterion.

### BRIEF DESCRIPTION OF THE DRAWINGS

The invention will now be described in a manner which does not limit the general inventive concept, with the aid of exemplary embodiments and by way of example with reference to the drawings, in which:

FIG. 1a shows a diagrammatic representation of a system of invention for predicting at least one cardiological dysfunction;

FIG. 1b shows the sinus rhythm of a heartbeat of an individual; and

FIGS. 2a-d shows ECG recordings, each with twelve ECG traces, each for four different individuals.

### DETAILED DESCRIPTION OF THE INVENTION

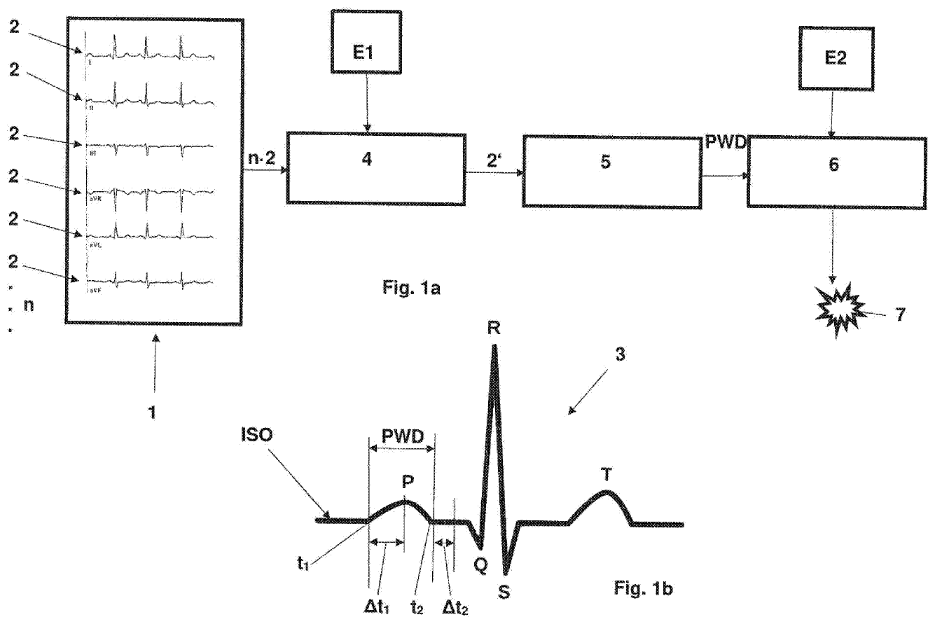

FIG. 1a diagrammatically shows the conformation of the components of the system of the invention for predicting at least one cardiological dysfunction in an individual. The means 1 for the preparation of a person's or individual's ECG, typically which is in the form of a digital n-lead ECG recording device, shows the electrical stimulation potential of the person's heart which can be associated with defined spatial cardiological regions, depending on the ECG trace. Standard ECG recording devices have a total of n=12 ECG traces 2 which have the following designations according to Einthoven, Goldberger and Wilson: I, II, III, aVR, aVL, aVF, V1 to V6.

Each of the individual ECG traces 2 has digitally recorded time signals which reflect the sinus rhythm 3 of the cardiac stimuli. FIG. 1b shows the conventional morphology of a sinus rhythm 3, which is composed of a chronological sequence of at least the P wave, a subsequent QRS complex as well as a finishing T wave. The system of the invention determines the P wave duration PWD of the person highly accurately, whereupon for the first time, dependable predictions regarding the risk of stroke in a person can be made with a reliability of at least 80%. In this regard, the chronological start t1 as well as the chronological end t2 of the P wave must be determined very accurately and without errors. This provides that the means 1 for the preparation of the ECG recorded for the individual provides the n ECG traces 2 at a scanning frequency of at least 500 Hz, which is preferably at least 1000 Hz. In the case of a scanning frequency of 1000 Hz, one time signal per millisecond can be acquired. If smaller temporal resolution is required, a digital electrocardiographic recording would have to be carried out at a correspondingly higher scanning frequency. Scanning frequencies of 2000 Hz or 5000 Hz are particularly desirable.

Furthermore, advantageously, the means 1 for the preparation of a person's ECG provides the n ECG traces 2 in an amplified form, in which the n ECG traces 2 are amplified by an amplification factor of at least 4, which preferably is at least 8, with respect to the signal level associated with the time signals, both in the time axis (x axis) as well as in the voltage-amplitude axis (y axis). Additionally, the amplification of the time axis is important in order to permit exact measurements. In this regard, the time axis speed is at least 100 mm/sec and preferably 200 mm/sec.

The n ECG traces 2 which have been digitally acquired and optionally processed in the aforementioned manner using signalling technology are fed to a computer-based selection means 4 in the form of a digital data set, which selects those ECG traces 2′ from the number n of ECG traces 2 on the basis of a first decision criterion E1, by means of which a chronological determination of the respective first point in time t1 as well as the second point in time t2 which is as accurate as possible can be made.

In order to determine the first point in time t1, which defines the start of the P wave, first, the isoelectric signal level ISO has to be determined. In a time domain which is located chronologically before the QRS complex, the start of the P wave corresponds to that point in time t1 at which the signal level of the time signal is distinguishable from the isoelectric signal level ISO by a technically verifiable signal level which rises positively from the isoelectric signal level. Preferably, this technically verifiable signal level is raised above the isoelectric signal level by twice the amount of the signal level. Directly at this technically verifiable signal level which is raised above the isoelectric signal level, the chronologically subsequent time signals which lie within a first time interval Δt1 immediately adjacent to the first point in time t1 must respectively have a positive increasing or, in the case of a reverse curve profile, negative reducing signal level. Typically, the first time interval corresponds to a maximum of half the P wave duration, in which the P wave increases positively. Preferably, the first time interval should therefore be between 40 ms and 80 ms.

In order to determine the second point in time t2, the time signals lying within a second time interval which is adjacent to the second point in time t2 should return to the isoelectric time signal level ISO. The isoelectric time interval Δt2 which is adjacent to the second point in time t2 should be at least 4 ms.

Advantageously again, the metrologically detected sinus rhythm is compared with a reference time signal model or a set of reference time signal models in the context of a software-supported pattern recognition. A pattern recognition of this type recognises the typical morphology of a sinus rhythm P wave within the respective metrologically detected ECG traces. If, however, the pattern recognition leads to a negative result, then the corresponding ECG trace is not suitable for accurately determining the chronological start as well as the chronological end of the P wave. Currently, the following characteristics are considered to be morphologically relevant criteria:
1. If the P wave has a monophase negative or negative-positive deflection in the I lead, then there is no sinus rhythm present and the analysis cannot be carried out.
2. If the P wave has a monophase positive deflection in the aVR lead, then there is no sinus rhythm present and the analysis cannot be carried out.
3. If the signal to noise ratio is more than 0.2 in one of the 12 leads even after averaging at least 2-1000 beats, then no analysis can be carried out in this lead.
4. The P wave morphology is compared within each of the 12 ECG traces among the 10 (to 1000) consecutively recorded P waves. The most frequent, that is dominant repetitive P wave morphology is determined. If the P wave morphology deviates from the “dominant” P wave morphology by more than 15% in at least one lead, then these defective morphologies are not used for the analysis.

The aforementioned cases provide criteria for carrying out the analysis, respectively in the absence of the criteria described in 1 and 2, and in order to exclude leads which are not suitable for the determination of the P wave duration from the analysis.

The following P wave characteristics are associated with the presence or absence of left atrial fibrosis (left atrial cardiomyopathy):
1. No suspicion of fibrosis in the left atrium (LA) is present if the sinus P wave morphology in the inferior II, III, aVF leads is monophase positive and the P wave duration is less than 143 ms in women and less than 154 ms in men.
2. Fibrosis in the left atrium (LA) is present if the total duration of the sinus P wave from the selected leads is more than 143 ms in women and more than 153 ms in men.
3. LA fibrosis is present if the sinus P wave morphology is positive-negative in two out of three II, III, aVF leads and in fact independent of the P wave duration.
4. A pronounced LA fibrosis is present if the sinus P wave morphology has a late P component. In these cases, there is a monophase positive P wave in the II, III, aVF leads which is followed by an isoelectric interval which in turn is followed by a chronologically offset P component in two of the following leads: I, aVL, aVR, V1-V6. The total P wave duration including the late component is as a rule more than 170 ms.

The ECG traces 2′ which are selected with the aid of the selection means 4, which may comprise at least two ECG traces, however a maximum of all n ECG traces, are fed to a processor-based analysis unit 5 which analyses the ECG traces 2′ in order to accurately determine the first and second points in time t1, t2. The respective chronological start as well as the chronological end of the P wave is accurately determined for all of the selected ECG traces 2′. Because of the time-synchronicity of all of the ECG traces, the respective earliest first point in time out of all of the first points in time determined from the respective selected ECG traces as well as the respective latest second point in time out of all of the second points in time determined from the respective selected ECG traces are determined. The respective earliest first as well as the latest second point in time define the actual chronological start as well as the chronological end of the P wave and therefore determine the exact P wave duration PWD. The exact P wave duration PWD determined in the context of the analysis unit 5 is fed to a comparator 6 which determines the discrepancy between the determined P wave duration PWD and a reference value and generates a signal 7 on the basis of a second decision criterion E2. In the case in which the determined P wave duration PWD is more than a maximum predetermined time interval Δtmax, then the comparator 6 generates the signal 7. Typically, the maximum time interval Δtmax is a region between 100 and 140 ms.

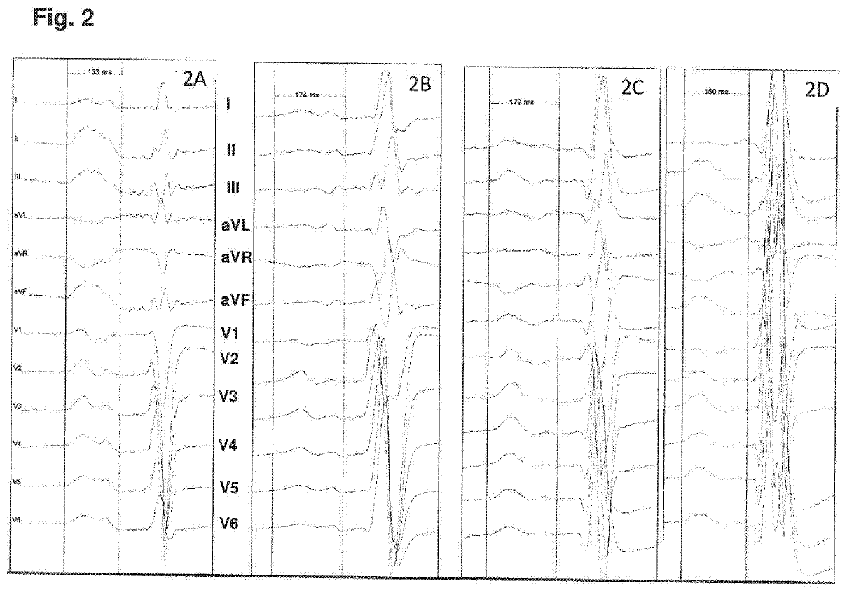

FIGS. 2a to d respectively show images from twelve-lead electrocardiograms from individuals with different levels of atrial cardiomyopathies. The individual ECG traces correspond to the following standard ECG leads: I, II, III, aVR, aVL, aVF, V1 to V6.

In the context of investigations carried out by the Applicant on patients with pulmonary vein isolation (PVI), in whom despite PVI, persistent atrial fibrillation (AF) occurs, it has become clear that arrhythmogenic slower conductive sites form or have been formed within and at the boundary regions of the left atrial low voltage substrate. Thus, an advanced left atrial low voltage substrate correlates with a reduced activation rate for the left atrium, that is electrical stimulation signal propagation, which is initiated at the sinus node and which propagates over the right and left atrium in the direction of the AV node, characterized by a longer duration of the P wave duration recorded with the aid of a 12-lead ECG.

FIGS. 2a to d show respective 12-lead surface ECGs of patients which are representative of different degrees of severity as regards the formation of arrhythmogenic fibrosis-rich slower conductive sites within the left atrial low voltage substrate. Furthermore, the tops of FIGS. 2a-d show the respective signal levels for two intracardiac catheter leads which respectively mark the actual chronological end of the P wave as a reference signal.

FIG. 2a shows the ECG of a non-critical patient with a heart with no noteworthy stimulation signal propagation delays in the left atrium without regions of scarring/fibrosis. The P wave is characterized in the II, III, aVF, V2-V6 ECG leads as a P wave with a normal morphology, in the form of a positive P wave. For an exact determination of the P wave duration, in this case 133 ms, the ECG II lead is used for determining the chronological start of the P wave, i.e. the first point in time t1, and the V4 ECG lead to establish the chronological end of the P wave, i.e. the second point in time.

FIG. 2b shows an ECG of a patient with the beginning of fibrotic tissue changes in the left atrium, which on the one hand lead to a change in the developing P wave morphology, by way of a multi-peaked wave profile. See the II, III, aVF ECG leads, as well as a lengthening of the P wave duration, in this case 174 ms. In this case, for an exact determination of the P wave duration, the I & V2 ECG leads serve for the determination of the chronological start of the P wave, that is the first point in time t1, as well as the V5 ECG lead for establishing the chronological end of the P wave, that is the second point in time.

FIG. 2c shows an ECG of a patient with advanced fibrotic tissue changes in the left atrium which lead to a decreasing stimulation signal propagation and above all to only poorly detectable signals, so that in many ECG traces, the clarity of the P wave in the left atrium is not detectable or only very poorly detectable. An exact analysis of the ECG leads shows up terminal P wave fractions in the I, aVL, V3-V6 leads which are only visualizable with substantial amplification. Here, for an exact determination of the P wave duration, the V4 ECG lead served for the determination of the chronological start of the P wave, that is the first point in time t1, as well as the I ECG lead for establishing the chronological end of the P wave, that is the second point in time. In this case, the P wave duration is 172 ms.

FIG. 2d shows an ECG of a patient with very advanced fibrotic tissue changes in the left atrium. This leads to a greatly reduced and weakly developed stimulation propagation. An exact analysis of the ECG leads shows P wave fractions in the I, V5 leads which are only visualizable with substantial amplification. Here, for an exact determination of the P wave duration, the V5 ECG lead serves for the determination of the chronological start of the P wave, that is the first point in time t1, as well as the I, V4-V6 ECG leads for establishing the chronological end of the P wave, that is the second point in time. In this case, the P wave duration is 160 ms.

The foregoing examples illustrate the difficulty and hence established necessity of accurately determining the P wave duration. As an example, in the case of a patient with an ECG in accordance with FIGS. 2c and 2d, a determination of the chronological end t2 of the P wave which was chronologically too early would lead to a seriously erroneous diagnosis, especially in those cases where time signals characterized by a low signal level chronologically after the monophase P wave configuration are not observed during a surface analysis, and so the P wave duration would be determined as being too short. In such a case, the diagnosis for the patient would be completely wrongly assessed as being of no risk to the health.

In the context of the digital signal evaluation on the basis of the analysis unit of the invention, which accurately evaluates the signal level of the time signals on signal level variations in the region of twice the signal level compared with the isoelectric signal level, atrial cardiac activities associated with the P wave can be accurately detected and be used as a basis for establishing the chronological end of the P wave.

### LIST OF REFERENCE NUMERALS

1 means for providing an ECG for an individual
2 ECG trace
3 sinus rhythm of a heartbeat
4 selection means
5 analysis unit
6 comparator
7 signal
2′ selected ECG traces
E1 first decision criterion
E2 second decision criterion
PWD pulse wave duration
ISO isoelectric signal level
t1 first point in time
t2 second point in time
Δt₁ first time interval
Δt₂ second time interval

1.-14. (canceled)

15. A system for predicting at least one cardiological dysfunction in an individual comprising:
means for preparing an ECG recording for an individual having a number n of time-synchronized ECG traces which each comprise a chronological sequence of time signals representing sinus rhythm of a heartbeat of the individual to which at least one P wave, a ventricular QRS complex and a T wave can be assigned in chronological order;
a selection means for selecting at least two ECG traces from the number n of time-synchronized ECG traces based on a first decision criterion;
an analysis unit which respectively analyses the selected ECG traces as follows:
a) determining an isoelectric signal level based on the chronological sequences of time signals from one of the at least two selected ECG traces;
b) determining a first point in time which is chronologically before the QRS ventricular complex from which time intervals of the selected at least two ECG traces have a signal level deviating from the isoelectric signal level;
c) determining a second point in time which chronologically follows the first point in time and which is chronologically before the QRS ventricular complex, at which, starting from a signal level, deviates from the isoelectric signal level at which the time signal for the selected ECG trace returns to the isoelectric signal level;
d) implementing the determining steps a) to c) for all of the selected at least two ECG traces;
e) determining an earliest first point in time from all of the first points in time determined from the selected at least two ECG traces and a latest second point in time from all second points in time determined from the selected at least two ECG traces; and
f) determining time intervals delimited by an earliest first point in time and latest second point in time corresponding to a known P wave duration; and
a comparator which determines a discrepancy between a known P wave duration and a reference value and which generates a signal based on a second decision criterion.

16. The system as claimed in claim 15, wherein:
the means for preparing of the ECG recording is a) a digital 12-lead ECG recording device, or is b) a storage medium in which the n ECG traces are stored in digital form, or is c) a body surface ECG recording system which has n multiple electrodes for recording electrical cardiac stimuli.

17. The system as claimed in claim 15, wherein:
the means for preparing the ECG of an individual provides the n ECG traces at a scanning frequency of at least 500 Hz.

18. The system as claimed in claim 16, wherein:
the scanning frequency is at least 1000 Hz.

19. The system as claimed in claim 15, wherein:
the means for preparing the ECG of an individual provides n amplified ECG traces, in which the n ECG traces which are recorded for the individual are amplified by an amplification factor of at least 4 with respect to the signal level associated with the time signals.

20. The system as claimed in claim 19, wherein:
the amplification factor is at least 8.

21. The system as claimed in claim 15, wherein:
prior to determination of step a), the analysis unit overlays and averages time signals from a sequence of at least two sinus rhythms of a heartbeat of the individual to obtain time-synchronized time signals for each selected ECG trace which represent a sinus rhythm of the heartbeat of the individual and which form a basis for performing steps a) to f).

22. The system as claimed in claim 15, comprising:
a reference time signal model of a sinus rhythm P wave forms a basis for the first decision criterion implemented by the selection means, which modeled sinus rhythm P wave is compared with the ECG traces by pattern recognition, and the selection means selects ECG traces aiding a pre-specified degree of similarity.

23. The system as claimed in claim 15, wherein:
the analysis unit evaluates the selected at least two ECG traces for determining the first and second points in time by steps of:
determining the first point in time at which the signal level of the time signal deviates from the isoelectric signal level to a positive larger signal level at which the time signals following a first time interval immediately adjacent to the first point in time have a positive increasing signal level; and
determining the second point in time at which the time signals which lie in a second time interval adjacent to the second point in time are at the isoelectric signal level or are delimited by starting of the ventricular complex.

24. The system as claimed in claim 23, wherein:
the first time interval is between 40 ms and 80 ms, and the second time interval measures at least 4 ms when the isoelectric signal level is present.

25. The system as claimed in claim 15, wherein:
the means for preparing an ECG recording comprises n=12 ECG leads corresponding to 12 standard ECG leads according to Einthoven, Goldberger and Wilson as follows:
I, II, III, aVR, aVL, aVF, V1-V6.

26. The system as claimed in claim 25, wherein:
the second decision criterion implemented by the selection means selects from the n ECG traces based on at least one of the following criteria; and
in order to determine the first point in time, at least two of the following ECG traces are taken into account:
II, III, aVF, aVR, V1, V2; and
in order to determine the second point in time, at least two of the following ECG traces are taken into account:
II, III, aVR, aVL, aVF, V2 to V6.

27. The system as claimed in claim 15, wherein:
the means for preparing the individual's ECG uses at least 3 ECG traces; and
the second decision criterion implemented in the selection means selects from the 3 ECG traces based on at least one of the following criteria:
determining the first and second points in time by at least two of the following ECG traces being taken into account:
inferior, lateral, inferolateral, superolateral or anterior ECG traces are associated with ECG electrodes respectively applied to opposite sides of a zero potential line of a field of the heart.

28. The system as claimed in claim 15, wherein:
when the second decision criterion is based on a maximum time interval Δtmax for which: 100≤Δtmax≤140 ms, and in which the determined P wave duration is more than Δtmax, the comparator generates the signal.

29. The system as claimed in claim 15, comprising:
an integrator for generating an integrated value based of the first and second points in time which are chronologically delimiting of determined P wave duration over a chronological sequence of time signals within the P wave duration; and
the comparator or another comparator compares the integrated value with a reference value and generates the signal based of a third decision criterion.

30. The system as claimed in claim 15, comprising:
an integrator for generating an integrated value based on the first and second points in time which are chronologically delimiting of determined P wave duration over a chronological sequence of the time signals within the P wave duration;
a divider for dividing a maximum or mean P wave amplitude from a P wave or a selected fraction thereof by a determined total P wave duration or a selected P wave fraction and determines a ratio thereof; and
the comparator or additional comparator compares the ratio with a reference value and generates the signal based on a third decision criterion.
